(19) United States
(12) Patent Application Publication
(10) Pub. No.: US 2015/0088010 A1
(43) Pub. Date: Mar. 26, 2015

(54) BLOOD PRESSURE CUFF APPARATUS AND SYSTEM

(71) Applicant: GENERAL ELECTRIC COMPANY, SCHENECTADY, NY (US)

(72) Inventor: Marvin Zellmer, New Berlin, WI (US)

(73) Assignee: GENERAL ELECTRIC COMPANY, SCHENECTADY, NY (US)

(21) Appl. No.: 14/035,417

(22) Filed: Sep. 24, 2013

(51) Int. Cl. A61B 5/022

(52) U.S. Cl. CPC A61B 5/02233
USPC 600/499

(57) ABSTRACT

A blood pressure cuff is disclosed herein. The blood pressure cuff includes a sleeve, a cuff bladder retained by the sleeve, and a retention feature secured to the sleeve. The retention feature is adapted to apply a force securing the blood pressure cuff to a patient.

### BACKGROUND OF THE INVENTION

The subject matter disclosed herein relates to a blood pressure cuff apparatus configured to minimize cuff migration or slip during a blood pressure measurement.

Conventional non-invasive blood pressure (NIBP) monitoring systems generally inflate a pressure cuff above the patient's systolic pressure and measure oscillations in the cuff as the cuff is deflated. The pressure cuff is wrapped around the patient's arm and secured thereto with a fastening mechanism such as, for example, a hook and loop fastening mechanism. After wrapping and securing the pressure cuff, a cuff bladder is inflated with air to apply a variable amount of pressure.

In order to maximize the precision with which a NIBP monitoring system measures a given patient's blood pressure, the cuff bladder should be centered over a predetermined target region. One problem is that the pressure cuff and cuff bladder can migrate or slip during the course of the blood pressure measurement. Such motion is particularly common with automated monitoring systems, and with NIBP measurements taken on a tapered limb. As an example, NIBP measurements taken on the upper arm of obese patients or muscular patients, and forearm NIPB measurements are all particularly prone to cuff migration.

### BRIEF DESCRIPTION OF THE INVENTION

The above-mentioned shortcomings, disadvantages and problems are addressed herein which will be understood by reading and understanding the following specification.

In an embodiment, a blood pressure cuff includes a sleeve, a cuff bladder retained by the sleeve, and a retention feature secured to the sleeve. The retention feature is adapted to apply a force securing the blood pressure cuff to a patient.

In another embodiment, a forearm blood pressure cuff includes an arcuate shaped sleeve, an arcuate shaped cuff bladder retained by the sleeve, and a retention feature secured to the sleeve. The retention feature is adapted to compress as the forearm blood pressure cuff is applied to a patient. This compression is adapted to generate a force securing the forearm blood pressure cuff to the patient and to thereby minimize migration of the forearm blood pressure cuff during a blood pressure measurement.

In another embodiment, a system for determining blood pressure includes a blood pressure monitor, and a blood pressure cuff pneumatically coupled with the blood pressure monitor. The blood pressure cuff includes a sleeve, a cuff bladder retained by the sleeve, and a retention feature secured to the sleeve. The retention feature is adapted to passively apply a force securing the blood pressure cuff to a patient and to thereby minimize migration of the blood pressure cuff during a blood pressure measurement.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of a non-invasive blood pressure monitoring system attached to a patient in accordance with an embodiment;

FIG. 2 is a schematic diagram of a pressure cuff in accordance with an embodiment;

FIG. 3 is an isometric diagram of the pressure cuff show in FIG. 2 in accordance with an embodiment;

FIG. 4 is a schematic diagram of a pressure cuff in accordance with another embodiment; and

FIG. 5 is a schematic diagram of a pressure cuff in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

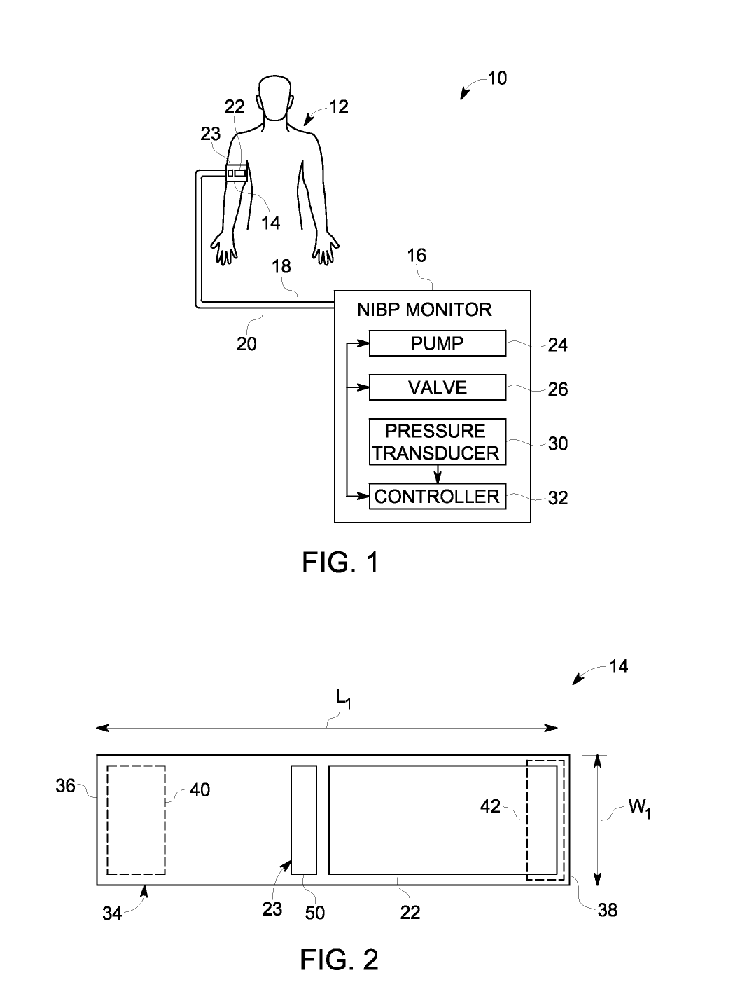

Referring to FIG. 1, a non-invasive blood pressure (NIBP) monitoring system 10 attached to a patient 12 is schematically shown in accordance with an embodiment. The NIBP monitoring system 10 includes a pressure cuff 14 pneumatically coupled with a NIBP monitor 16 via the flexible tubes 18, 20. The pressure cuff 14 includes a cuff bladder 22 and a retention feature 23. For purposes of this disclosure, the term bladder should be defined to include an inflatable pocket or chamber. The NIBP monitor 16 includes a pump 24 adapted to inflate the cuff bladder 22, and one or more valves 26 adapted to deflate the cuff bladder 22. The NIBP monitor 16 also includes a pressure transducer 30 operable to sense or identify pressure pulses at the portion of the limb to which the pressure cuff 14 is attached. A controller 32 converts the pressure pulse data from the pressure transducer 30 into blood pressure data in a known manner.

It is well known to those skilled in the art that optimal NIBP monitoring precision generally requires that cuff bladder 22 remain centered over a predetermined target region of interest, typically an artery. Accordingly, the retention feature 23 is adapted to minimize pressure cuff 14 migration or slip away from the target region of interest during the course of NIBP measurement, and to thereby optimize NIBP monitoring system 10 precision.

The NIBP monitor 16 is configured to measure mean arterial pressure (MAP), systolic blood pressure (SYS), and/or diastolic blood pressure (DIA) by inflating the pressure cuff 14 to a supra-systolic pressure level and measuring oscillations under the pressure cuff 14 as the pressure cuff 14 is deflated. For purposes of this disclosure, the term “oscillation” refers to a measurable pressure level pulse produced by a change in volume of an artery under the pressure cuff 14.

Referring to FIG. 2, a schematic illustration of the pressure cuff 14 is shown in accordance with an embodiment. Common reference numbers from FIG. 1 will be used to identify common components.

The pressure cuff 14 comprises a flexible, non-distensible sleeve 34 and the cuff bladder 22. The sleeve 34 is flexible such that it may be conveniently wrapped around a patient's limb, and non-distensible such that it generally does not expand or swell in response to pressure. According to one embodiment, the sleeve 34 comprises two or more layers that are impermeable to air and are fused together near their peripheral edges in a manner adapted to form the cuff bladder 22. According to another embodiment, the cuff bladder 22 is a separate component retained by the sleeve 34. The sleeve 34 is generally rectangular defining a sleeve end 36 and a generally opposite sleeve end 38. A cuff length L1 is defined between sleeve ends 34 and 36, and a cuff width W1 is defined in a direction perpendicular thereto. The cuff bladder 22 is also generally rectangular.

The sleeve 34 is preferably long enough to be wrapped around a patient's limb such that the sleeve ends 36, 38 overlap each other by an amount necessary to secure the pressure cuff 14. According to one embodiment, the sleeve 34 comprises complementary hook and loop type fastening portions 40, 42 adapted to retain the pressure cuff 14 on the patient.

The retention feature 23 is shown in accordance with an embodiment as comprising a rectangular pad 50. The pad 50 is preferably composed of a compressible material having a memory (e.g., foam or elastomer) such that it generally returns to its original shape after deformation. This material compression applies a retention force adapted to resist migration or slip of the pressure cuff 14 during an NIBP measurement. By minimizing slip, the cuff bladder 22 remains more precisely centered over a target region of interest to thereby optimize measurement precision.

The stiffness of the pad 50 may be selected to allow for a measured or selectable retention force when exposed to forces typically encountered during the process of applying the pressure cuff 14 to the target limb. Advantageously, the retention force applied in this manner is passive meaning a user can produce it without any taking additional steps in the process of applying the pressure cuff 14. In other words, the act of wrapping and securing the pressure cuff 14 in the traditional manner has the added effect of generating the retention force. It should also be appreciated that the pressure cuff 14 with the pad 50 is generally inexpensive to manufacture and assemble.

The pad 50 is preferably secured to the sleeve 34 between ends 36, 38 at a position away from the cuff bladder 22; however other positions can be envisioned. It should be appreciated that NIBP analysis involves the measurement of oscillations under the cuff bladder 22 during deflation, and that anything positioned between the cuff bladder 22 and the patient could interfere with such measurement. Accordingly, by positioning the pad 50 away from the cuff bladder 22, system interference is minimized. According to an alternate embodiment (not shown), the pad 50 may be positioned on the cuff bladder 22. For this embodiment, pad 50 composition is preferably selected to minimize pressure measurement interference and may include, for example, open-cell foam.

The pad 50 may be oriented to extend in a direction along the cuff width W1 in order to axially align with a target limb when secured thereto; however other orientations can be envisioned. A pad 50 positioned in this manner advantageously applies a generally uniform force across the entire width of the pressure cuff 14 when applied to a patient. A force applied in this manner is well suited to securing the pressure cuff 14 in place and thereby minimizing slip.

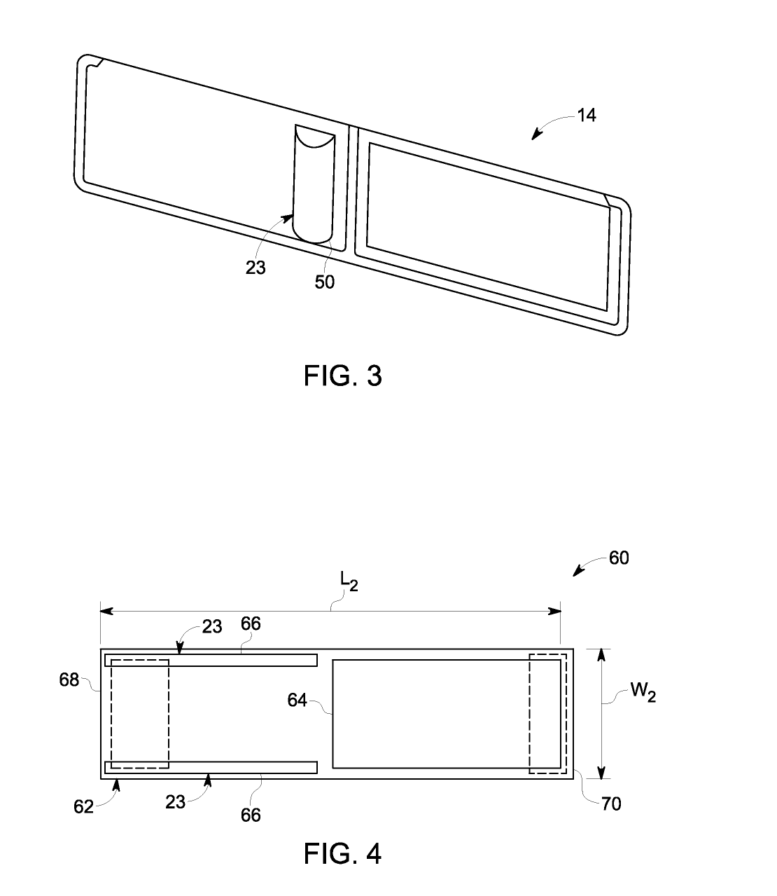

Referring to FIG. 3, an isometric illustration of the pressure cuff 14 is shown in accordance with an embodiment. The depicted embodiment of the pad 50 defines a thickness protruding away from the surface of the sleeve 34. This thickness may define a semi-cylindrical geometry such that the pad 50 is thickest near its centerline. It should be appreciated that other pad 50 geometries may be envisioned.

Referring to FIG. 4, a schematic illustration of a pressure cuff 60 is shown in accordance with an embodiment. The pressure cuff 60 comprises a sleeve 62 and a cuff bladder 64. The sleeve 62 and cuff bladder 64 are similar in composition and operation to the sleeve 34 and cuff bladder 22 (shown in FIG. 2), respectively. The pressure cuff 60 also comprises a retention feature 23 shown in accordance with an embodiment as comprising one or more flexible inserts 66.

The sleeve 62 is generally rectangular defining a sleeve end 68 and a generally opposite sleeve end 70. A cuff length L2 is defined between sleeve ends 68 and 70, and a cuff width W2 is defined in a direction perpendicular thereto. The cuff bladder 64 is also generally rectangular.

The flexible inserts 66 are preferably composed of a material having a memory (e.g., plastic) such that they generally return to their original shape after deformation. The flexible inserts 66 are preferably elongated members oriented to extend along the cuff length L2 of the pressure cuff 60 such that the flexible inserts 66 elastically deform and coil around a target limb as the pressure cuff 60 is applied to a patient. As the flexible inserts 66 coil, they apply an outward retention force tending to uncoil the pressure cuff 60 thereby effectively tightening the cuff wrap and further securing the pressure cuff 60 to the target limb. This retention force helps minimize migration or slip of the pressure cuff 60 during an NIBP measurement. By minimizing slip, the cuff bladder 64 remains more precisely centered over a target region of interest to thereby optimize measurement precision.

The stiffness of the flexible inserts 66 may be selected to allow for a measured or selectable retention force when exposed to forces typically encountered during the process of applying the pressure cuff 60 to the target limb. As described previously with respect to the pad 50 (shown in FIG. 2), the retention force produced by the flexible inserts 66 is passive. It should also be appreciated that the pressure cuff 60 with the flexible inserts 66 is generally inexpensive to manufacture and assemble.

According to the depicted embodiment, there are two flexible inserts 66 retained at a position away from the cuff bladder 22 in order to minimize NIBP measurement interference; however other quantities and positions can be envisioned. The two flexible inserts 66 may be disposed such that one is positioned near the top of the non-bladder side of the sleeve 62, oriented to extend along the length of the cuff bladder 64. The other flexible insert 66 may be positioned near the bottom of the non-bladder side of the sleeve 62, oriented to extend along the length of the cuff bladder 64. In this manner, when the pressure cuff 60 is wrapped around a target limb, the two flexible inserts 66 may apply a securing force at opposing ends of the pressure cuff 60 to retain the cuff bladder 64 therebetween.

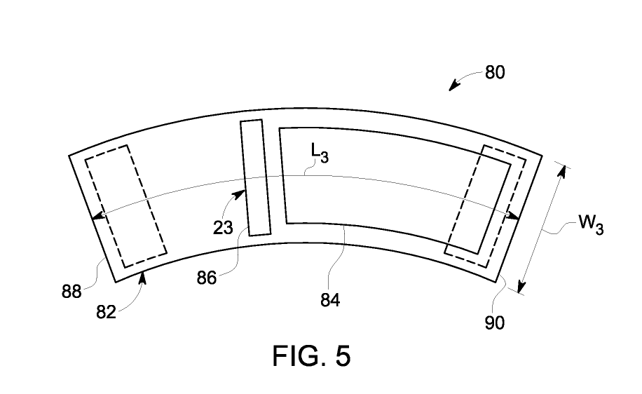

Referring to FIG. 5, a schematic illustration of a pressure cuff 80 is shown in accordance with an embodiment. The pressure cuff 80 comprises a sleeve 82 and a cuff bladder 84. The sleeve 82 and cuff bladder 84 function similarly to the sleeve 34 and cuff bladder 22 (shown in FIG. 2), but differ in their shape. The pressure cuff 80 also comprises a retention feature 23 shown in accordance with an embodiment as comprising a pad 86.

The sleeve 82 is generally arcuate shaped defining a sleeve end 88 and a generally opposite sleeve end 90. A cuff length L3 is defined between sleeve ends 88 and 90, and a cuff width W3 is defined in a direction generally perpendicular thereto. The cuff bladder 84 is also generally arcuate shaped.

The arcuate shape of the sleeve 82 forms a generally conical shape when the sleeve ends 88 and 90 are engaged in the manner described hereinabove with respect to the pressure cuff 14 (shown in FIG. 2). This conical shape is intended to conform with and thereby accommodate a patient's forearm. Applying the pressure cuff 80 to the forearm is particularly advantageous for obese patients and muscular patients for whom more traditional rectangular NIBP cuffs may not fit well.

It should be appreciated that the potential for NIBP cuff migration or slip away from the clinically appropriate location is increased for tapered limbs. In other words, a pressure cuff is more likely to slip when applied to a tapered limb such as a patient's forearm. The pad 86 is therefore particularly well suited for use with forearm cuffs.

The pad 86 is similar in composition (e.g., a compressible material having a memory) and operation to the pad 50 (shown in FIG. 2). This material compression applies a retention force adapted to resist migration or slip of the pressure cuff 80 during an NIBP measurement.

The stiffness of the pad 86 may be selected to allow for a measured or selectable retention force when exposed to forces typically encountered during the process of applying the pressure cuff 14 to the target limb. As described previously with respect to the pad 50 (shown in FIG. 2), the retention force produced by the pad 86 is passive. It should also be appreciated that the pressure cuff 80 with the pad 86 is generally inexpensive to manufacture and assemble.

The pad 86 is preferably secured to the sleeve 82 between ends 88, 90 at a position away from the cuff bladder 84 in order to minimize measurement interference. The pad 86 may be oriented to extend in a direction along the width of the sleeve 82 in order to axially align with a patient's forearm, and to thereby apply a generally uniform force across the entire width W3 of the pressure cuff 80.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. A blood pressure cuff comprising:
a sleeve;
a cuff bladder retained by the sleeve; and
a retention feature secured to the sleeve;
wherein the retention feature is adapted to apply a force securing the blood pressure cuff to a patient.

2. The blood pressure cuff of claim 1, wherein the sleeve and the cuff bladder are generally rectangular shaped.

3. The blood pressure cuff of claim 1, wherein the sleeve and the cuff bladder are arcuate shaped.

4. The blood pressure cuff of claim 1, wherein the retention feature comprises a compressible pad adapted to passively apply the force securing the blood pressure cuff to the patient.

5. The blood pressure cuff of claim 4, wherein the compressible pad is secured to the sleeve in a manner adapted to apply a generally uniform force across the width of the sleeve when the blood pressure cuff is applied to a patient.

6. The blood pressure cuff of claim 4, wherein the compressible pad comprises an open cell foam material to minimize blood pressure measurement interference.

7. The blood pressure cuff of claim 4, wherein the compressible pad is secured to the sleeve at a position away from the cuff bladder to minimize blood pressure measurement interference.

8. The blood pressure cuff of claim 1, wherein the retention feature comprises a flexible insert adapted to elastically deform and coil around a target limb when the blood pressure cuff is applied to a patient.

9. The blood pressure cuff of claim 1, wherein the flexible insert includes a plurality of plastic flexible inserts.

10. The blood pressure cuff of claim 1, wherein the cuff bladder is defined by the sleeve.

11. The blood pressure cuff of claim 1, wherein the cuff bladder is disposed within the sleeve.

12. A forearm blood pressure cuff comprising:
an arcuate shaped sleeve;
an arcuate shaped cuff bladder retained by the sleeve; and
a retention feature secured to the sleeve;
wherein the retention feature is adapted to compress as the forearm blood pressure cuff is applied to a patient, and wherein the compression is adapted to generate a force securing the forearm blood pressure cuff to the patient and to thereby minimize migration of the forearm blood pressure cuff during a blood pressure measurement.

13. The forearm blood pressure cuff of claim 12, wherein the retention feature is secured to the sleeve in a manner adapted to apply a generally uniform force across the width of the sleeve when the blood pressure cuff is applied to a patient.

14. The blood pressure cuff of claim 12, wherein the retention feature comprises an open cell foam pad adapted minimize blood pressure measurement interference.

15. The forearm blood pressure cuff of claim 12, wherein the retention feature is secured to the sleeve at a position away from the cuff bladder to minimize blood pressure measurement interference.

16. The forearm blood pressure cuff of claim 12, wherein the cuff bladder is defined by the sleeve.

17. The forearm blood pressure cuff of claim 12, wherein the cuff bladder is disposed within the sleeve.

18. A system for determining blood pressure comprising:
a blood pressure monitor; and
a blood pressure cuff pneumatically coupled with the blood pressure monitor, said blood pressure cuff comprising:
a sleeve; and
a cuff bladder retained by the sleeve; and
a retention feature secured to the sleeve;
wherein the retention feature is adapted to passively apply a force securing the blood pressure cuff to a patient and to thereby minimize migration of the blood pressure cuff during a blood pressure measurement.

19. The system of claim 18, wherein the retention feature comprises a compressible open-cell foam pad secured to the sleeve in a manner adapted to produce a generally uniform force across the width of the sleeve when the blood pressure cuff is applied to a patient.

20. The system of claim 18, wherein the retention feature comprises a plurality of flexible inserts adapted to elastically deform and coil around a target limb when the blood pressure cuff is applied to the patient.
